(19) United States
(12) Plant Patent Application Publication
(10) Pub. No.: US 2004/0055064 P1
(43) Pub. Date: Mar. 18, 2004

(54) CALLA LILY PLANT NAMED 'BLACK STAR'

(76) Inventor: Cecilius Jan-Jochem Randag, Schagen (NL)

Correspondence Address: Russell D. Orkin, 700 Koppers Building, 436 Seventh Avenue, Pittsburgh, PA, 15219-1818, US

(21) Appl. No.: 10/241,892

(22) Filed: Sep. 12, 2002

(51) Int. Cl. A01H 5/00

(52) U.S. Cl. PLT/263

(57) ABSTRACT

‘Black Star’ is a new variety of calla lily having a flower with a purple/black colored spathe and a purple/black colored spadix and producing 1-4 flowers per tuber. The flower stems are red and about 50 cm in height. The leaves are green with whitish spots and have a leathery texture. On the edge of the leaves there is a red stripe. The petioles have a red to black color.

### BOTANICAL CLASSIFICATION

Zantedeschia sprengeri

### VARIETAL DENOMINATION

‘Black Star’

### BACKGROUND OF THE NEW PLANT

The present invention comprises a new and distinct cultivar of calla lily, botanically known as Zantedeschia sprengeri and hereinafter referred to by the cultivar name ‘Black Star’.

‘Black Star’ is a product of a planned breeding program which had the objective of creating Zantedeschia hybrids for cut flower production in a wide range of colors with a large, classic flower shape. The breeding program began in 1989 and the new cultivar is a seedling selected from the cross of a Zantedeschia sprengeri variety known as ‘Schwarzwalder’ with a Zantedeschia sprengeri seedling. ‘Black Star’ was originated in 1998 by the inventor Cecilius Jan-Jochem Randag in 't Zand, The Netherlands as one flowering plant within the progeny of the stated cross.

The first act of asexual reproduction of ‘Black Star’ by tissue culture was performed by the inventor in August 2000 in 't Zand, The Netherlands. Subsequent asexual reproduction by tissue culture at the same location has demonstrated that the combination of characteristics as herein disclosed for the new cultivar are retained through successive generations of asexual reproduction.

The following traits have been repeatedly observed and determined to be basic characteristics of ‘Black Star’ which, in combination, distinguish this calla lily as a new and distinct cultivar:

1. Deep purple spathe;

2. Deep purple spadix;

3. Red flower stem;

4. Red edge on the leaves; and

5. Red/black petiole of the leaves.

### BRIEF DESCRIPTION OF THE DRAWINGS

The accompanying photographic drawings illustrate the new variety, with the colors being as nearly true as is possible with color illustrations of this type.

FIG. 1 is a photograph of the whole plant of ‘Black Star’;

FIG. 2 is a photograph of the young leaf of the ‘Black Star’ plant;

FIG. 3 is a photograph of the petiole;

FIG. 4 is a photograph showing the uniformity of plants produced in tissue culture; and

FIG. 5 is a photograph showing the stability of the characteristics after tissue culture.

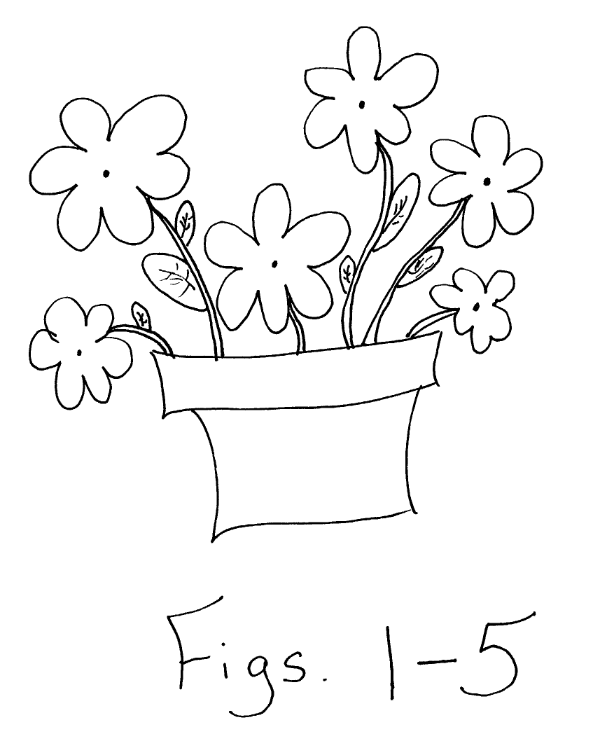

### DESCRIPTION OF THE NEW PLANT

The following observations, measurements, and comparisons describe plants grown in 't Zand, The Netherlands under greenhouse conditions which approximate those generally used in horticulture practice. Color references are made to the R.H.S. Colour Chart of The Royal Horticultural Society of London, except where general color terms of ordinary significance are used.

### The Plant

Size:

Height of the leaf canopy above the soil.—50-65 cm.

Height of top of inflorescence above the soil.—60-75 cm.

Diameter.—30-40 cm.

Form.—Erect.

Number of inflorescences per tuber size:

Length 9-12 cm.—0-1 inflorescences.

Length 12-15 cm.—1-3 inflorescences.

Length 15-18 cm.—3-4 inflorescences.

Branches:

Character.—Medium.

Color.—Between 143A and 144A with spots of 53A.

Leaves:

Size.—Width: 11-15 cm. Length: 20-30 cm.

Number per plant.—4-10.

Shape.—Elliptic.

Color.—Upper surface: Between 137A and 137B. Lower surface: Between 137A and 137B.

Margin.—Wavy with red line.

Veins.—Configuration: Pinnate. Color: Between 144A and 53A.

Surface quality.—Leathery.

Petiole:

Length.—30-50 cm.

Color.—Base is 53A, upper part varies between 53A and 202A.

Roots:

Color.—White.

Branching.—Average amount.

### The Flower

Spathe:

Size.—Length: 8-10 cm. Width: 5-7 cm.

Color.—Upper surface: Varies between 187A and 202A. Lower surface: Varies between 187A and 202A.

Veins.—Color: Varies between 187A and 202A.

Shape.—Cupped.

Spadix:

Size.—Length: 20-45 mm. Diameter: 3-7 mm.

Color.—187A.

Position relative to spathe.—Upright.

Peduncle:

Size.—Length: 50-65 cm. Diameter: 8-10 mm.

Color.—Varies between 53A and 202A.

Reproductive organs:

Location of female organs.—Basal position of the spadix.

Location of male organs.—Upper position of the spadix.

Perianth.—Conspicuous.

Stamens.—Not visible before pollen release.

Pistil.—Length beyond perianth: About 1 mm.

Flowering: Tubers planted in a greenhouse in The Netherlands in February produce flowers in April. Tubers planted outdoors in The Netherlands in May produce flowers in July.

Lastingness: About three weeks on the plant.

Fragrance: None.

Disease/pest resistance: No unusual susceptibility to diseases or pests has been noted to date.

I claim:

1. A new and distinct variety of calla lily named ‘Black Star’ as described and illustrated.
